(19) United States
(12) Patent Application Publication
(10) Pub. No.: US 2023/0293777 A1
(43) Pub. Date: Sep. 21, 2023

(54) IMPLANT MATERIALS AND IMPLANTS SUITABLE FOR IMPLANTATION IN BONE DEFECT REPAIR AND SPINAL FUSION, AND PREPARATION METHODS THEREOF

(71) Applicant: THE FIRST AFFILIATED HOSPITAL SUN YAT-SEN UNIVERSITY, Guangzhou, Guangdong (CN)

(72) Inventors: Xuenong ZOU, Guangzhou, Guangdong (CN); Hualin YI, Guangzhou, Guangdong (CN); Jun CHEN, Guangzhou, Guangdong (CN); Gang WANG, Guangzhou, Guangdong (CN); Yan CHEN, Guangzhou, Guangdong (CN); Hao HU, Guangzhou, Guangdong (CN)

(21) Appl. No.: 18/021,051

(22) PCT Filed: Jul. 13, 2021
(86) PCT No.: PCT/CN2021/105956

§ 371 Date: Feb. 13, 2023

(30) Foreign Application Priority Data
Aug. 13, 2020 (CN) 202010812292.4
Aug. 13, 2020 (CN) 202021687163.9

(51) Int. Cl. A61L 27/54; A61L 27/52; A61L 27/10; A61L 27/34; A61F 2/44; A61F 2/30

(52) U.S. Cl. CPC A61L 27/54; A61L 27/52; A61L 27/10; A61L 27/34; A61F 2/4455; A61F 2/3094; A61F 2002/2835

(57) ABSTRACT

Provided is an implant material, an implant suitable for implantation in bone defect repair and in spinal fusion, and a preparation method thereof. The implant material is used for filling in an internal cavity of a titanium cage/titanium stent (10) or a bone defect site, and is consisted of an upper layer (21), a lower layer (22) and an intermediate layer between the upper layer (21) and lower layer (22), wherein the intermediate layer is composed of an annular coating region (23) located at the periphery and a central region (24) located inside the annular coating region. The central region (24) is filled with a first filling material, which is a gel material having a pro-osteogenic effect. The annular coating region (23) is filled with a second filling material, which is a gel material regulating epigenetics. The upper layer (21) and lower layer (22) are filled with a third filling material, which is a gel material regulating immuno-inflammatory response and stress response. The implant of the present disclosure can adapt to the microenvironment of different regions of the injury sites and orderly regulate the repair process, and is suitable for implantation in bone defect repair and spinal fusion.

### CROSS-REFERENCE TO RELATED APPLICATIONS

This application claims priority to PCT Application No. PCT/CN2021/105956, having a filling date of Jul. 13, 2021, which is based on Chinese Application No. 202010812292.4, having a filing date of Aug. 13, 2020, and Chinese Application No. 202021687163.9, having a filing date of Aug. 13, 2020, the entire contents all of which are incorporated herein by reference.

### TECHNICAL FIELD

The following relates to the technical field of implants for implantation in bone defect repair and in spinal fusion surgery.

### BACKGROUND

At present, surgical implant materials for transplantation in bone defect repair and in spinal fusion surgery are usually hollow metallic frameworks (titanium cage or titanium stent), filled with autologous fractured bone tissues or other allogeneic osteogenic materials; or the implant materials are directly autologous or allogeneic bone pieces supplemented with osteogenic materials, for implanting and filling in the middle part of the defect to fill the scattered autologous fractured bone tissues or other allogeneic osteogenic materials in surgery. In recent years, since tissue engineering technology has progressed, a variety of materials has been used for transplantation in bone defect repair and spinal fusion surgery, including hydroxyapatite, active bone cement, and so on.

As the complex injury microenvironment needs to be precisely adjusted in the process of bone defect repair and spinal fusion, different materials may respond to the injury microenvironment to different degrees, and impact on the efficiency of bone tissue regeneration to different degrees. Most of the existing implant materials have a holistic structure, without regional characteristics. Thus, these implant materials are difficult to be adapted to the microenvironment of different regions in injury sites, and cannot easily and orderly regulate the repair process, thereby having limitations in promoting osteogenic effects. Especially, central regions of the implant materials are far away from the bone tissues and thus lack a corresponding osteogenic microenvironment. The low oxygen environment formed by the materials is unfavorable for migrating osteoblasts to the central regions to colonize and differentiate, and further forming new bone tissues and the corresponding trabecular bone structures. And there is no intelligent and precise adjustment process for microenvironment response of the injury sites and the osteogenic repair process.

In view of this, it is necessary to develop new surgical implant materials that can solve the above problems.

### SUMMARY OF THE INVENTION

An aspect relates to an implant for implantation in bone defect repair and in spinal fusion, which can be adapted to the microenvironment of different regions in the injury sites and orderly regulate the repair process.

In order to achieve the above purpose, the present disclosure adopts the following technical solutions:

An implant material suitable for implantation in bone defect repair and in spinal fusion, is characterized in that, the implant material is used for filling in an internal cavity of a titanium cage (titanium stent) or a bone defect site, and is consisted of an upper layer, a lower layer and an intermediate layer between the upper and lower layers, wherein the intermediate layer is composed of an annular coating region located at the periphery and a central region located inside the annular coating region; the central region is filled with a first filling material, which is a gel material having a pro-osteogenic effect; the annular coating region is filled with a second filling material, which is a gel material regulating epigenetics.; the upper and lower layers are filled with a third filling material, which is a gel material regulating immuno-inflammatory response and stress response.

Further, the first filling material is composed of a self-healing hydrogel and bioactive glass coated by the self-healing hydrogel, wherein the bioactive glass is loaded with mRNA and miRNA having a chemotactic effect and a pro-osteogenic effect.

In one embodiment, the mRNA and miRNA having a chemotactic effect and a pro-osteogenic effect is MCP1 or IL8, or a mixture thereof.

Further, the second filling material is composed of a self-healing hydrogel, bioactive glass and nano-lipoid, wherein the bioactive glass and nano-lipoid are coated by the self-healing hydrogel, the bioactive glass is loaded with inflammatory regulation-related mRNA/protein and osteogenesis-related and angiogenesis-related mRNA and protein, and the nano-lipoid is loaded with osteogenesis signal-related nucleic acids/protein molecules regulating epigenetics.

In one embodiment, the inflammatory regulation-related mRNA/protein and osteogenesis-related and angiogenesis-related mRNA and protein are one or a mixture of more selected from the group consisted of RUNX2, OSX, BMP2/BMP7, TGFB1, FGF2, and VEGF. The osteogenesis signal-related nucleic acids and protein molecules regulating epigenetics are one or more RNA-lipoid complex(es) selected from the group consisted of miR424, miR146, and miR200a.

Further, the third filling material is composed of a self-healing hydrogel and bioactive glass coated by the self-healing hydrogel, wherein the bioactive glass is loaded with hypoxia stress and inflammation regulation-related mRNA/protein molecules.

In one embodiment, the hypoxia stress and inflammation regulation-related mRNA/protein molecules are HIF-1 or timp1, or a mixture thereof.

In one embodiment, the self-healing hydrogel comprises 10 wt %-15 wt % of gelatin methacrylate, 2 wt %-8 wt % of oxidized dextran, 2.5 wt %-10 wt % of gelatin, and 0.1-0.5 wt % of Irgacure 2959-photoinitiator.

Another aspect relates to an implant suitable for implantation in bone defect repair and in spinal fusion, comprising a titanium cage or titanium stent and any one of the above-mentioned implant material filled in an internal cavity of the titanium cage.

Another aspect relates to a method for preparing the above-mentioned implant material suitable for implantation in bone defect repair and in spinal fusion.

In order to achieve this purpose, the present disclosure adopts the following technical scheme: a method for preparing any an above-mentioned implant material suitable for implantation in bone defect repair and in spinal fusion, comprising following steps:
(1) preparing bioactive glass;
(2) preparing nano-lipoid;
(3) preparing self-healing hydrogel;
(4) mixing the self-healing hydrogel with the bioactive glass, or mixing the self-healing hydrogel with bioactive glass and nano-lipoid, to prepare a first filling material, a second filling material, and a third filling material, respectively.

According to the structural features of the bone defect sites, the present disclosure designs an implant material having a multi-layer structure, which can respond to the injury microenvironment and the bone repair process. According to the regional characteristics of the injury sites to be repaired, the implant material is divided into three different regions, and each of the regions has different gel materials and thus has different functions. By taking advantage of the differences in material components of the different regions, the implant material for transplantation in bone defect repair and in spinal fusion can respond to different regional characteristics of the injury microenvironment, and manage the process of cellular migration and new bone formation in bone tissues through the spatial structure, thereby achieving intelligent and precise regulation of biomaterials on bone tissues.

### BRIEF DESCRIPTION OF THE DRAWINGS

Some of examples will be described in detail, with reference to the following figures, wherein like designations denote like members, wherein:

FIG. 1 depicts a structural schematic diagram of the implant of the present disclosure; and

FIG. 2 depicts a structural schematic diagram of the filler in an implant suitable for spinal fusion.

titanium cage (titanium stent) 10, filler 20, upper layer 21, lower layer 22, annular coating region 23, central region 24.

### DETAILED DESCRIPTION OF THE EMBODIMENT

According to the structural features of the bone defect sites, the present disclosure designs an implant material having multi-layer/region structure, which can respond to the injury microenvironment and the bone repair process. The implant material is used for filling in an internal cavity of a titanium cage (titanium stent) or a bone defect site. The implant material, as the most basic structure, is consisted of an upper layer, a lower layer and an intermediate layer between the upper and lower layers, wherein the intermediate layer is composed of an annular coating region located at the periphery and a central region located inside the annular coating region. The central region is filled with a first filling material, which is a gel material having a pro-osteogenic effect. The annular coating region is filled with a second filling material, which is a gel material regulating epigenetics. The upper and lower layers are filled with a third filling material, which is a gel material regulating immuno-inflammatory response and stress response.

Considering the convenience of actual application, the first filling material, the second filling material and the third filling material are all wrapped in a packaged bag or a sealed bottle. The volumes of the first filling material, the second filling material and the third filling material are respectively set according to different types of the titanium cages.

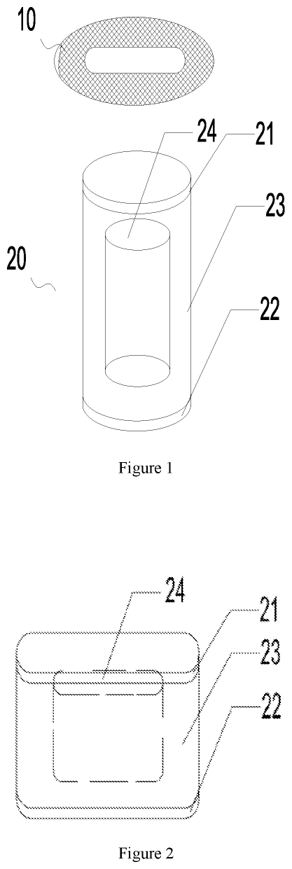

Considering the convenience of use, the present disclosure also provides a holistic implant, comprising a titanium cage and an implant material. As shown in FIG. 1, the implant, suitable for implantation in bone defect repair and in spinal fusion, comprises a titanium cage (titanium stent) 10 and a filler 20 filled in an internal cavity of the titanium cage. The filler 20 is comprised of an upper layer 21, a lower layer 22 and an intermediate layer between the upper layer and the lower layer. The intermediate layer is composed of an annular coating region 23 located at the periphery and a central region 24 located inside the annular coating region. The central region is filled with a first filling material, the annular coating region is filled with a second filling material, and the upper and lower layers are filled with a third filling material.

In addition to a basic function, i.e. pro-osteogenic effect, there are other functional differences among the upper/lower layer, the annular coating region and the central region. The main differences are as following: the upper/lower layer contacts with bone tissue and mainly responds to inflammatory response and stress regulation, while the annular coating region 23 in the intermediate layer regulates osteogenesis by epigenetics, and the central region 24 increases chemotactic signals. The above functions can be achieved in different regions by various gel ingredients and various coated contents in such regions.

The bioactive glass is loaded with pro-osteogenic factors, and nucleic acid or protein molecule which regulates hypoxia stress and inflammatory response. The nano-lipoid is loaded with nucleic acid or protein molecule which is related to osteogenic signals and has epigenetics regulation effect. The self-healing hydrogel is proportionally mixed with the loaded bioactive glass and the loaded nano-lipoid to form gel filling materials in different layers/regions. The details are as follows in Table 1.

TABLE 1
Ingredients and contents

| | Bioactive glass | Nano-lipoid | Self-healing hydrogel | Mixing ratio |
|---|---|---|---|---|
| First | micro-nano bioactive | None | 10 wt % to 15 wt % of | bioactive glass |
| filling | glass, loaded with | | gelatin methacrylate, 2 | and self-healing |
| material | osteogenesis-related and | | wt % to 8 wt % of | hydrogel in a |
| | angiogenesis-related | | oxidized dextran, 2.5 | mass ratio of |
| | regulatory | | wt % to 10 wt % of | 10% to 15% |
| | mRNA/proteins, and with | | gelatin, and 0.1 to 0.5 | |
| | cellular chemotaxis | | wt % of Irgacure 2959- | |
| | regulation-related | | photoinitiator | |
| | proteins, such as MCP1 | | | |
| | or IL8. | | | |
| Second | micro-nano bioactive | RNA-lipoid | 10 wt % to 15 wt % of | bioactive glass |
| filling | glass, loaded with | complexes | gelatin methacrylate, 2 | and self-healing |
| material | inflammatory regulation- | activating | wt % to 8 wt % of | hydrogel in a |
| | related mRNA/proteins, | osteogenesis | oxidized dextran, 2.5 | mass ratio of |
| | and with | signaling | wt % to 10 wt % of | 10% to15%; |
| | osteogenesis-related and | pathway, such | gelatin, and 0.1 to 0.5 | nano-lipoid and |
| | angiogenesis-related | as epigenetic | wt % of Irgacure 2959- | self-healing |
| | mRNA and proteins, such | regulators, | photoinitiator | hydrogel in a |
| | as RUNX2, OSX, | miR424, | | mass ratio of |
| | BMP2/BMP7, TGFB1, | miR146, | | 0.1% to 1% |
| | FGF2, VEGF, etc. | miR200a, etc. | | |
| Third | micro-nano bioactive | None | 10 wt % to 15 wt % of | bioactive glass |
| filling | glass, loaded with hypoxia | | gelatin methacrylate, 2 | and self-healing |
| material | stress-related and | | wt % to 8 wt % of | hydrogel in a |
| | inflammation-related | | oxidized dextran, 2.5 | mass ratio of |
| | regulatory factor | | wt % to 10 wt % of | 5% to 10% |
| | mRNA/proteins, such as | | gelatin, and 0.1 to 0.5 | |
| | HIF-1, timp1, etc. | | wt % of Irgacure 2959- | |
| | | | photoinitiator | |

The preparing steps of the present disclosure are as follows:
1. Preparing bioactive glass, loaded with nucleic acids or proteins having a pro-osteogenic effect (such as, proteins promoting osteogenesis, e.g., BMP-2, and microRNA or mRNA regulating bone formation).
2. Preparing nano-lipoid, loaded with stress-regulation nucleic acids and proteins (such as, immunosuppressive factors and hypoxia stress regulatory factors, e.g., IL-10, HIF-la, as well as microRNA or mRNA with immunoregulatory effects).
3. Preparing self-healing hydrogel, whose ingredients are prepared according to the ratio in Table 1. Firstly, the gelatin and oxidized dextran form a hydrogel that can automatically repair the bone defects, through a reversible Schiff base reaction. Then, gelatin methacrylate is cross-linked by UV light to form a second network structure, which enhances the mechanical strength of the hydrogel. Finally, a double-network self-healing hydrogel with high mechanical strength is prepared.
4. Mixing the self-healing hydrogel with bioactive glass and nano-lipoid, loaded with nucleic acids and proteins, according to the ratios in Table 1, to prepare the first filling material, the second filling material and the third filling material, respectively.
5. Filling the first filling material, the second filling material and the third filling material into the corresponding bone defect sites by injection; alternatively, after a freeze-dried treatment for the self-healing hydrogels of the first filling material, the second filling material, and the third filling material, filling the dried hydrogels into the titanium cage for spinal fusion or into the bone defect sites as a whole, and then the dried hydrogels contacting with the bone tissues to absorb water and expand, and finally shaping them under UV irradiation.

According to specific situations, in some cases, it can be selected to only inject the first filling material and the third filling material to repair the bone defect sites.

According to the differences of microenvironment in the bone repair sites, the repair sites can be divided into a proximal region contacting with bone tissues at both sides and a distal region far away from bone tissue at center. As the proximal region is directly contacted with the bone tissues, the implant materials participate in the hypoxic stress response of the injury environment immediately and the migrating cells firstly respond to early bone repair events. In contrast, as the distal region far away from bone tissues is at the center of the implant, the migrating cells are subjected to more intense stress, and thus the osteogenesis process is relatively delayed. According to the different temporal and spatial characteristics of different osteogenic repair microenvironments, different injection sequences can be selected. For example, the third filling material, which regulates immuno-inflammatory response and stress responses, is firstly injected into the proximal region contacting with bone tissue, namely upper and lower layers. Then, the first filling material, which contains bioactive glass, is injected into the central region of the internal cavity of the titanium cage, wherein the bioactive glass is loaded with mRNA/protein having chemotactic effect and pro-osteogenic effect. Finally, the second filling material, which regulates epigenetics, is injected around the first filling material to form an annular coating region. For the titanium cage, the second filling material can be injected into the titanium cage to form the an annular coating region, and then the first filling material can be injected into the titanium cage to form a core of the osteogenic material in the central region, and finally the third filling material can be injected into contacted regions, i.e., the upper layer/lower layer which are contacted with bone tissues. In some instances, the first filling material, the second filling material, and the third filling material can also be injected in sequence.

Using the self-healing property of the double-network hydrogel stent, the microenvironment of the filling regions can be controlled and adjusted, thereby achieving the purpose of support and bone repair.

### Example 1

A filler of an implant suitable for implantation in spinal fusion, as shown in FIG. 2, was prepared.

I. Preparation of a first filling material.

1.1 Construction of bioactive glass-loaded protein molecules having pro-osteogenic effect by a chemical reaction of a phosphate group in nucleic acid molecules.

The ingredients comprised bioactive glass, which was loaded with osteogenesis-related and angiogenesis-related regulatory protein, e.g., VEGF (10 ng/ml), and cell chemotaxis regulation-related protein, e.g., MCP1 (2 ng/ml).

1.2 Preparation of a double-network self-healing hydrogel system with Gelatin Methacrylate (gelMA).

The ingredients comprised gelatin methacrylate, oxidized dextran, gelatin, Irgacure 2959-photoinitiator, dissolved in PBS respectively, mixed and stirred well. The final concentration ratio (w/v) was as follows: 12% of gelatin methacrylate, 4% of oxidized dextran, 5% of gelatin, and 0.3% of photoinitiator.

1.3 The bioactive glass prepared in Step 1.1 was mixed and cross-linked with the self-healing hydrogel prepared in Step 1.2 at a concentration of 10% of the bioactive glass to produce the first filling material.

II. Preparation of a second filling material.

2.1 Construction of bioactive glass-loaded mRNA/protein molecules having pro-osteogenic effect by a chemical reaction of a phosphate group in nucleic acid molecules.

The ingredients comprised micro-nano bioactive glass loaded with mRNA expressing inflammation-related regulator IL10 (2 ng/ml), and micro-nano bioactive glass loaded with osteogenesis-related and angiogenesis-related mRNA, including RUNX2 (200 μg/ml), BMP2 (500 μg/ml), TGFB1 (10 μg/ml), and VEGF (100 μg/ml).

2.2 Preparation of a basic structural unit, nano-lipoid loaded with miRNA molecule, and the outer layer of the nano-lipoid was coated with alginate gel having a sustained release effect.

The ingredients comprised miRNA-lipoid complex, which was formed by miR146 (200 μg/ml) that activated the osteogenic signaling pathway and nano-lipoid, and the outer layer of the miRNA-lipoid complex was coated with 2% of alginate gel.

2.3 Preparation of the double-network self-healing hydrogel system with gelMA.

The ingredients comprised gelatin methacrylate, oxidized dextran, gelatin, Irgacure 2959-photoinitiator, dissolved in PBS respectively, mixed and stirred well. The final concentration ratio (w/v) was as follows: 15% of gelatin methacrylate, 2% of oxidized dextran, 2.5% of gelatin, and 0.5% of photoinitiator.

2.4 The bioactive glass prepared in Step 2.1 and the basic structural unit, i.e., the nano-lipoid loaded with miRNA molecules, prepared in Step 2.2 were mixed with the self-healing hydrogel prepared in Step 2.3, in a proportion of 10% of bioactive glass and 1% of nano-lipoid particles, to prepare the second filling material.

III. Preparation of a third filling material.

3.1 Construction of bioactive glass-loaded protein molecules having a pro-osteogenic effect by a chemical reaction of phosphate group in nucleic acid molecules.

The ingredients comprised micro-nano bioactive glass, loaded with hypoxia stress and inflammation-related regulatory factor HIF-1 (10 ng/ml).

3.2 Preparation of the double-network self-healing hydrogel system with gelMA.

The ingredients comprised gelatin methacrylate, oxidized dextran, gelatin, Irgacure 2959-photoinitiator, dissolved in PBS respectively, mixed and stirred well. The final concentration ratio (w/v) was as follows: 14% of gelatin methacrylate, 3% of oxidized dextran, 3.75% of gelatin, and 0.4% of photoinitiator.

3.3 The bioactive glass prepared in Step 3.1 was evenly mixed with the self-healing hydrogel prepared in Step 3.2, in a proportion of 5% of bioactive glass, to prepare the third filling material.

IV. The third filling material, namely the hydrogel containing 5% of bioactive glass, loaded with inflammation regulatory factor, was injected into contact sites, i.e., the upper layer/lower layer of the titanium cage, which were contacted with the bone tissues to form a complete implant material framework structure.

V. The first filling material, namely a gel mixture containing bioactive glass, which was loaded with mRNA having a pro-osteogenic effect and chemokines and further coated by the double-network self-healing hydrogel, was injected into the titanium cage to form a core of the osteogenic material in the central region.

VI. The second filling material, namely a gel mixture containing bioactive glass, which was loaded with mRNA having pro-osteogenic effect in combination with epigenetic regulators and further coated by the double-network self-healing hydrogel, was injected into the titanium cage to form an annular coating region.

### Example 2

A filler suitable for implantation in lumbar fusion was prepared, and it might be in a shape as shown in FIG. 1.

This example had identical regions to those in Example 1, but was prepared in a different external shape, as shown in FIG. 1, and the filling materials in some parts of these regions may be loaded with ingredients in a different proportion.

I. Preparation of a first filling material.

1.1 Construction of bioactive glass-loaded protein molecules having pro-osteogenic effect by a chemical reaction of phosphate group in nucleic acid molecules.

The ingredients comprised bioactive glass, which was loaded with osteogenesis-related and angiogenesis-related regulatory protein, VEGF (10 ng/ml), and cell chemotaxis regulation-related protein, e.g., TIMP1 (2 ng/ml).

1.2 Preparation of a double-network self-healing hydrogel system with gelMA.

The ingredients comprised gelatin methacrylate, oxidized dextran, gelatin, Irgacure 2959-photoinitiator, dissolved in PBS respectively, mixed and stirred well. The final concentration ratio (w/v) was as follows: 12% of gelatin methacrylate, 4% of oxidized dextran, 5% of gelatin, and 0.3% of photoinitiator.

1.3 The bioactive glass prepared in Step 1.1 was mixed and cross-linked with the self-healing hydrogel prepared in Step 1.2 at a concentration of 15% of the bioactive glass to produce the first filling material.

II. Preparation of a second filling material.

2.1 Construction of bioactive glass-loaded mRNA/protein molecules having pro-osteogenic effectby a chemical reaction of phosphate group in nucleic acid molecules.

The ingredients comprised micro-nano bioactive glass loaded with mRNA expressing inflammation-related regulator IL10 (2 ng/ml), and micro-nano bioactive glass loaded with osteogenesis-related and angiogenesis-related mRNA, including OSX (200 μg/ml), BMP2 (500 μg/ml), and VEGF (200 μg/ml).

2.2 Preparation of a basic structural unit, nano-lipoid loaded with miRNA molecules, and the outer layer of the nano-lipoid was coated with alginate gel having a sustained release effect.

The ingredients comprised miRNA-lipoid complex, which was formed by miR424 (100 μg/ml) and miR200a (200 μg/ml) that activated the osteogenic signaling pathway and nano-lipoid, and the outer layer of the miRNA-lipoid complex was coated with 2% of alginate gel.

2.3 Preparation of the double-network self-healing hydrogel system with gelMA.

The ingredients comprised gelatin methacrylate, oxidized dextran, gelatin, Irgacure 2959-photoinitiator, dissolved in PBS respectively, mixed and stirred well. The final concentration ratio (w/v) was as follows: 15% of gelatin methacrylate, 2% of oxidized dextran, 2.5% of gelatin, and 0.5% of photoinitiator.

2.4 The bioactive glass prepared in Step 2.1 and the basic structural unit, i.e., the nano-lipoid loaded with miRNA molecules, prepared in Step 2.2 were mixed with the self-healing hydrogel prepared in Step 2.3, in a proportion of 15% of bioactive glass and 0.1% of nano-lipoid particles, to prepare the second filling material.

III. Preparation of a third filling material.

3.1 Construction of bioactive glass-loaded protein molecules having a pro-osteogenic effect by a chemical reaction of phosphate group in nucleic acid molecules.

The ingredients comprised micro-nano bioactive glass, loaded with hypoxia stress and inflammation-related regulatory factor HIF-1 (10 ng/ml).

3.2 Preparation of the double-network self-healing hydrogel system with gelMA.

The ingredients comprised gelatin methacrylate, oxidized dextran, gelatin, Irgacure 2959-photoinitiator, dissolved in PBS respectively, mixed and stirred well. The final concentration ratio (w/v) was as follows: 14% of gelatin methacrylate, 3% of oxidized dextran, 3.75% of gelatin, and 0.4% of photoinitiator.

3.3 The bioactive glass prepared in Step 3.1 was evenly mixed with the self-healing hydrogel prepared in Step 3.2, in a proportion of 10% of bioactive glass, to prepare the third filling material.

IV. The third filling material, namely the hydrogel containing 10% of bioactive glass, loaded with inflammation regulatory factor, was injected into contact sites, i.e., the upper layer/lower layer of the titanium cage, which were contacted with the bone tissues to form a complete implant material framework structure.

V. The first filling material, namely a gel mixture containing bioactive glass, which was loaded with mRNA having a pro-osteogenic effect and chemokines and further coated by the double-network self-healing hydrogel, was injected into the titanium cage to form a core of the osteogenic material in the central region.

VI. The second filling material, namely a gel mixture containing bioactive glass, which was loaded with mRNA having pro-osteogenic effect in combination with epigenetic regulators and further coated by the double-network self-healing hydrogel, was injected into the titanium cage to form an annular coating region.

Although the present invention has been disclosed in the form of preferred embodiments and variations thereon, it will be understood that numerous additional modifications and variations could be made thereto without departing from the scope of the invention.

For the sake of clarity, it is to be understood that the use of ‘a’ or ‘an’ throughout this application does not exclude a plurality, and ‘comprising’ does not exclude other steps or elements.

1. An implant material suitable for implantation in bone defect repair and in spinal fusion, wherein the implant material is used for filling in an internal cavity of a titanium cage/titanium stent or a bone defect site, and is consisted of an upper layer, a lower layer and an intermediate layer between the upper and lower layers, wherein the intermediate layer is composed of an annular coating region located at the periphery and a central region located inside the annular coating region; the central region is filled with a first filling material, which is a gel material having a pro-osteogenic effect; the annular coating region is filled with a second filling material, which is a gel material regulating epigenetics; the upper layer and the lower layer are filled with a third filling material, which is a gel material regulating immuno-inflammatory response and stress response.

2. The implant material suitable for implantation in bone defect repair and in spinal fusion according to claim 1, wherein the first filling material is composed of a self-healing hydrogel and bioactive glass coated by the self-healing hydrogel, wherein the bioactive glass is loaded with mRNA and miRNA having a chemotactic effect and a pro-osteogenic effect.

3. The implant material suitable for implantation in bone defect repair and in spinal fusion according to claim 2, wherein the mRNA and miRNA having a chemotactic effect and a pro-osteogenic effect is MCP1 or IL8, or a mixture thereof.

4. The implant material suitable for implantation in bone defect repair and in spinal fusion according to claim 1, wherein the second filling material is composed of a self-healing hydrogel, bioactive glass and a nano-lipoid, wherein the bioactive glass and the nano-lipoid are coated by the self-healing hydrogel, the bioactive glass is loaded with inflammatory regulation-related mRNA/protein and osteogenesis-related and angiogenesis-related mRNA and protein, and the nano-lipoid is loaded with an osteogenesis signal-related nucleic acid and protein molecule regulating epigenetics.

5. The implant material suitable for implantation in bone defect repair and in spinal fusion according to claim 4, wherein the inflammatory regulation-related mRNA/protein and the osteogenesis-related and angiogenesis-related mRNA and protein are one or a mixture of more selected from the group consisted of RUNX2, OSX, BMP2/BMP7, TGFB1, FGF2, and VEGF; the osteogenesis signal-related nucleic acid and protein molecules regulating epigenetics are one or more RNA-lipoid complex selected from the group consisted of miR424, miR146, and miR200a.

6. The implant material suitable for implantation in bone defect repair and in spinal fusion according to claim 1, wherein the third filling material is composed of a self-healing hydrogel and bioactive glass coated by the self-healing hydrogel, wherein the bioactive glass is loaded with hypoxia stress and inflammation regulation-related mRNA/protein molecules.

7. The implant material suitable for implantation in bone defect repair and in spinal fusion according to claim 6, wherein the hypoxia stress and inflammation regulation-related mRNA/protein molecules are one or a mixture of more selected from the group of HIF-1, or TIMP1, or other hypoxia stress and inflammation regulation-related factors.

8. The implant material suitable for implantation in bone defect repair and in spinal fusion according to claim 1, wherein the self-healing hydrogel comprises 10 wt % to 15 wt % of gelatin methacrylate, 2 wt % to 8 wt % of oxidized dextran, 2.5 wt % to 10 wt % of gelatin, and 0.1 wt % to 0.5 wt % of Irgacure 2959-photoinitiator.

9. An implant suitable for implantation in bone defect repair and in spinal fusion, comprising a titanium cage/titanium stent and the implant material according to any one claim 1, filled in an internal cavity of the titanium cage.

10. A method for preparing the implant material suitable for implantation in bone defect repair and in spinal fusion according to claim 1, comprising following steps:
(1) preparing bioactive glass;
(2) preparing nano-lipoid;
(3) preparing self-healing hydrogel;
(4) mixing the self-healing hydrogel with the bioactive glass, or mixing the self-healing hydrogel with bioactive glass and nano-lipoid, to prepare a first filling material, a second filling material, and a third filling material, respectively.

11. The implant suitable for implantation in bone defect repair and in spinal fusion according to claim 9, wherein the first filling material is composed of a self-healing hydrogel and bioactive glass coated by the self-healing hydrogel, wherein the bioactive glass is loaded with mRNA and miRNA having a chemotactic effect and a pro-osteogenic effect.

12. The implant suitable for implantation in bone defect repair and in spinal fusion according to claim 11, wherein the mRNA and miRNA having a chemotactic effect and a pro-osteogenic effect is MCP1 or IL8, or a mixture thereof.

13. The implant suitable for implantation in bone defect repair and in spinal fusion according to claim 9, wherein the second filling material is composed of a self-healing hydrogel, bioactive glass and a nano-lipoid, wherein the bioactive glass and the nano-lipoid are coated by the self-healing hydrogel, the bioactive glass is loaded with inflammatory regulation-related mRNA/protein and osteogenesis-related and angiogenesis-related mRNA and protein, and the nano-lipoid is loaded with an osteogenesis signal-related nucleic acid and protein molecule regulating epigenetics.

14. The implant suitable for implantation in bone defect repair and in spinal fusion according to claim 13, wherein the inflammatory regulation-related mRNA/protein and the osteogenesis-related and angiogenesis-related mRNA and protein are one or a mixture of more selected from the group consisted of RUNX2, OSX, BMP2/BMP7, TGFB1, FGF2, and VEGF; the osteogenesis signal-related nucleic acid and protein molecules regulating epigenetics are one or more RNA-lipoid complex selected from the group consisted of miR424, miR146, and miR200a.

15. The implant suitable for implantation in bone defect repair and in spinal fusion according to claim 9, wherein the third filling material is composed of a self-healing hydrogel and bioactive glass coated by the self-healing hydrogel, wherein the bioactive glass is loaded with hypoxia stress and inflammation regulation-related mRNA/protein molecules.

16. The method for preparing the implant material suitable for implantation in bone defect repair and in spinal fusion according to claim 10, wherein the first filling material is composed of a self-healing hydrogel and bioactive glass coated by the self-healing hydrogel, wherein the bioactive glass is loaded with mRNA and miRNA having a chemotactic effect and a pro-osteogenic effect.

17. The method for preparing the implant material suitable for implantation in bone defect repair and in spinal fusion according to claim 16, wherein the mRNA and miRNA having a chemotactic effect and a pro-osteogenic effect is MCP1 or IL8, or a mixture thereof.

18. The method for preparing the implant material suitable for implantation in bone defect repair and in spinal fusion according to claim 10, wherein the second filling material is composed of a self-healing hydrogel, bioactive glass and a nano-lipoid, wherein the bioactive glass and the nano-lipoid are coated by the self-healing hydrogel, the bioactive glass is loaded with inflammatory regulation-related mRNA/protein and osteogenesis-related and angiogenesis-related mRNA and protein, and the nano-lipoid is loaded with an osteogenesis signal-related nucleic acid and protein molecule regulating epigenetics.

19. The method for preparing the implant material suitable for implantation in bone defect repair and in spinal fusion according to claim 18, wherein the inflammatory regulation-related mRNA/protein and the osteogenesis-related and angiogenesis-related mRNA and protein are one or a mixture of more selected from the group consisted of RUNX2, OSX, BMP2/BMP7, TGFB1, FGF2, and VEGF; the osteogenesis signal-related nucleic acid and protein molecules regulating epigenetics are one or more RNA-lipoid complex selected from the group consisted of miR424, miR146, and miR200a.

20. The method for preparing the implant material suitable for implantation in bone defect repair and in spinal fusion according to claim 10, wherein the third filling material is composed of a self-healing hydrogel and bioactive glass coated by the self-healing hydrogel, wherein the bioactive glass is loaded with hypoxia stress and inflammation regulation-related mRNA/protein molecules.
